(19) United States
(12) Patent Application Publication
(10) Pub. No.: US 2021/0186436 A1
(43) Pub. Date: Jun. 24, 2021

(54) JOINT DISORDER RISK EVALUATION DEVICE, SYSTEM, METHOD, AND PROGRAM

(71) Applicant: NEC Corporator, Minato-ku, Tokyo (JP)

(72) Inventor: Kenichiro FUKUSHI, Tokyo (JP)

(73) Assignee: NEC Corporator, Minato-ku, Tokyo (JP)

(21) Appl. No.: 17/265,926

(22) PCT Filed: Aug. 7, 2018
(86) PCT No.: PCT/JP2018/029565

§ 371 Date: Feb. 4, 2021

(51) Int. Cl. A61B 5/00; A61B 5/11

(52) U.S. Cl. CPC A61B 5/7275; A61B 5/4585; A61B 5/112; A61B 2562/0247; A61B 5/4566; A61B 2562/0219; A61B 5/4595

(57) ABSTRACT

A presymptomatic disease countermeasure system 90 includes a motion measurement unit 91 which obtains motion data that is time-series data representing motion, by measuring the motion of an object, a joint reaction force computation unit 92 which computes joint reaction force at a joint to be evaluated among joints of the object, using the obtained motion data and ground reaction force data that is time-series data representing ground reaction force applied to the object, a feature amount computation unit 93 which computes a feature amount representing a load repeatedly applied to the joint to be evaluated on the basis of the computed joint reaction force, and a determination unit 94 which determines a joint disorder risk indicator that is an indicator representing a joint disorder risk that is the risk of causing joint disorder, on the basis of the computed feature amount.

### TECHNICAL FIELD

This invention relates to a joint disorder (an antiarthropathic) risk evaluation device, a joint disorder risk evaluation system, a presymptomatic disease countermeasure system, a joint disorder risk evaluation method, a presymptomatic disease countermeasure method, and a joint disorder risk evaluation program.

### BACKGROUND ART

The patent literature 1 describes a technique for evaluating risk of developing a joint disorder. The patent literature 1 describes a gait analysis method for determining the risk of occurrence of knee osteoarthritis using a knee adduction moment and a knee abduction moment estimated from acceleration signals measured by an accelerometer attached to a near part of a tibia and a near part of a heel.

The patent literature 2 describes a support system that can improve walking motion of a subject in a simple configuration and accurately. Hereinafter, the risk of developing a joint disorder is referred to as a joint disorder risk.

In addition, the non patent literature 1 describes a major cause and so on of osteoarthritis.

### CITATION LIST

### Patent Literature

PTL1: Japanese Patent Application Laid-Open No. 2017-202236
PTL2: Japanese Patent Application Laid-Open No. 2011-041752

### Non Patent Literature

Perry, J. and Burnfield, J. M., “Gait Analysis: Normal and Pathological Function, 2nd Edition”, Ishiyaku Publishers, Inc., Mar. 1, 2012, pp. 200-201

### SUMMARY OF INVENTION

### Technical Problem

The non patent literature 1 describes that “there was little increase in the friction coefficient of the knee joint during 1 hour of standing holding a heavy load”. In other words, the reaction force (hereinafter, referred to as the joint reaction force) applied to the joint during stand straight without moving is large but constant. Therefore, the joint disorder risk caused by the joint reaction force is not high during stand straight without moving.

In addition, the non patent literature 1 describes that the joint disorder risk, such as osteoarthritis, etc., is increased due to degeneration of an articular cartilage when instantaneous loads are repeatedly applied to the a joint.

However, the gait analysis method described in the patent literature 1 does not take into account a load repeatedly applied to the joint. Therefore, there is a problem that accuracy of the gait analysis method described in the patent literature 1 is low in evaluating the joint disorder risk. Further, the support system described in the patent literature 2 also does not take into account a load repeatedly applied to the joint.

### Objective of the Invention

Therefore, it is an object of the present invention to provide a joint disorder risk evaluation device, a joint disorder risk evaluation system, a presymptomatic disease countermeasure system, a joint disorder risk evaluation method, a presymptomatic disease countermeasure method, and a joint disorder risk evaluation program that solve the above described problem and enable a more accurate evaluation of the joint disorder risk.

### Solution to Problem

The joint disorder risk evaluation device according to the present invention is characterized by including a joint reaction force computation unit which computes joint reaction force at a joint to be evaluated among joints of an object, using motion data that is time-series data representing motion of the object, and ground reaction force data that is time-series data representing ground reaction force applied to the object, a feature amount computation unit which computes a feature amount representing a load repeatedly applied to the joint to be evaluated on the basis of the computed joint reaction force, and a determination unit which determines a joint disorder risk indicator that is an indicator representing a joint disorder risk that is the risk of causing joint disorder, on the basis of the computed feature amount.

The joint disorder risk evaluation system according to the present invention is characterized by including a motion measurement unit which obtains motion data that is time-series data representing motion, by measuring the motion of an object, a joint reaction force computation unit which computes joint reaction force at a joint to be evaluated among joints of the object, using the obtained motion data and ground reaction force data that is time-series data representing ground reaction force applied to the object, a feature amount computation unit which computes a feature amount representing a load repeatedly applied to the joint to be evaluated on the basis of the computed joint reaction force, and a determination unit which determines a joint disorder risk indicator that is an indicator representing a joint disorder risk that is the risk of causing joint disorder, on the basis of the computed feature amount.

The presymptomatic disease countermeasure system according to the present invention is characterized by including a motion measurement unit which obtains motion data that is time-series data representing motion, by measuring the motion of an object, a joint reaction force computation unit which computes joint reaction force at a joint to be evaluated among joints of the object, using the obtained motion data and ground reaction force data that is time-series data representing ground reaction force applied to the object, a feature amount computation unit which computes a feature amount representing a load repeatedly applied to the joint to be evaluated on the basis of the computed joint reaction force, a determination unit which determines a joint disorder risk indicator that is an indicator representing a joint disorder risk that is the risk of causing joint disorder, on the basis of the computed feature amount, and an output unit which outputs the determined joint disorder risk indicator together with countermeasures for preventing development of symptoms caused by the computed joint reaction force.

The joint disorder risk evaluation method according to the present invention is characterized by computing joint reaction force at a joint to be evaluated among joints of an object, using motion data that is time-series data representing motion of the object, and ground reaction force data that is time-series data representing ground reaction force applied to the object, computing a feature amount representing a load repeatedly applied to the joint to be evaluated on the basis of the computed joint reaction force, and determining a joint disorder risk indicator that is an indicator representing a joint disorder risk that is the risk of causing joint disorder, on the basis of the computed feature amount.

The joint disorder risk evaluation method according to the present invention is characterized by obtaining motion data that is time-series data representing motion, by measuring the motion of an object, computing joint reaction force at a joint to be evaluated among joints of the object, using the obtained motion data and ground reaction force data that is time-series data representing ground reaction force applied to the object, computing a feature amount representing a load repeatedly applied to the joint to be evaluated on the basis of the computed joint reaction force, and determining a joint disorder risk indicator that is an indicator representing a joint disorder risk that is the risk of causing joint disorder, on the basis of the computed feature amount.

The presymptomatic disease countermeasure method according to the present invention is characterized by obtaining motion data that is time-series data representing motion, by measuring the motion of an object, computing joint reaction force at a joint to be evaluated among joints of the object, using the obtained motion data and ground reaction force data that is time-series data representing ground reaction force applied to the object, computing a feature amount representing a load repeatedly applied to the joint to be evaluated on the basis of the computed joint reaction force, determining a joint disorder risk indicator that is an indicator representing a joint disorder risk that is the risk of causing joint disorder, on the basis of the computed feature amount, and outputting the determined joint disorder risk indicator together with countermeasures for preventing development of symptoms caused by the computed joint reaction force.

A joint disorder risk evaluation program according to the present invention is characterized by causing a computer to perform a joint reaction force computation process of computing joint reaction force at a joint to be evaluated among joints of an object, using motion data that is time-series data representing motion of the object, and ground reaction force data that is time-series data representing ground reaction force applied to the object, a feature amount computation process of computing a feature amount representing a load repeatedly applied to the joint to be evaluated on the basis of the computed joint reaction force, and a determination process of determining a joint disorder risk indicator that is an indicator representing a joint disorder risk that is the risk of causing joint disorder, on the basis of the computed feature amount.

### Advantageous Effects of Invention

According to the present invention, the joint disorder risk can be evaluated with higher accuracy.

### BRIEF DESCRIPTION OF DRAWINGS

FIG. 1 is a block diagram showing an example configuration of a first exemplary embodiment of a joint disorder risk evaluation system according to the present invention.

FIG. 2 is an explanatory diagram showing an example of motion data of a knee joint.

FIG. 3 is an explanatory diagram showing an example of motion data of an ankle joint.

FIG. 4 is an explanatory diagram showing an example of ground reaction force data.

FIG. 5 is a block diagram showing an example configuration of the joint disorder risk evaluation device 300 of the first exemplary embodiment.

FIG. 6 is an explanatory diagram showing an example of a joint disorder risk indicator table of the first exemplary embodiment.

FIG. 7 is a flowchart showing an operation of a joint disorder risk evaluation process by the joint disorder risk evaluation device 300 of the first exemplary embodiment.

FIG. 8 is a block diagram showing an example configuration of the second exemplary embodiment of a presymptomatic locomotive syndrome (musculoskeletal deterioration) countermeasure system according to the present invention.

FIG. 9 is an explanatory diagram showing an example of the second exemplary embodiment of the presymptomatic locomotive syndrome countermeasure system 30.

FIG. 10 is a flowchart showing an operation of a presymptomatic disease countermeasure method displaying process by the presymptomatic locomotive syndrome countermeasure system 30 of the second exemplary embodiment.

FIG. 11 is an explanatory diagram showing an example hardware configuration of the joint disorder risk evaluation device 300 according to the present invention.

FIG. 12 is a block diagram showing an outline of the joint disorder risk evaluation device according to the present invention.

FIG. 13 is a block diagram showing an outline of the joint disorder risk evaluation system according to the present invention.

FIG. 14 is a block diagram showing an outline of the presymptomatic disease countermeasure system.

### DESCRIPTION OF EMBODIMENTS

### Exemplary Embodiment 1

### [Explanation of Configuration]

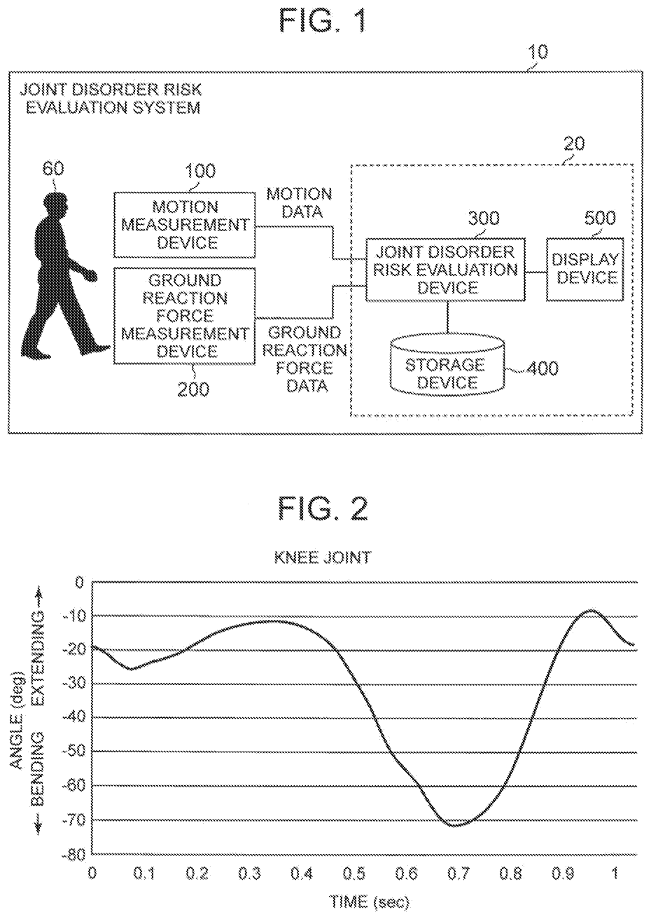

The exemplary embodiments of the present invention will be described with reference to the drawings. FIG. 1 is a block diagram showing an example configuration of a first exemplary embodiment of a joint disorder risk evaluation system according to the present invention. The joint disorder risk evaluation system 10 shown in FIG. 1 is a system for evaluating a joint disorder risk of a pedestrian 60.

As shown in FIG. 1, the joint disorder risk evaluation system 10 includes a motion measurement device 100, a ground reaction force measurement device 200, a joint disorder risk evaluation device 300, a storage device 400, and a display device 500.

The means of connection between the devices included in the joint disorder risk evaluation system 10 is a wired connection using, for example, a LAN (Local Area Network) cable or a USB (Universal Serial Bus) cable.

The connection means between the devices may be a wireless connection using Bluetooth (registered trademark), Wi-Fi (registered trademark), or the like. The means of connection between devices included in the joint disorder risk evaluation system 10 of this exemplary embodiment is not particularly limited.

In FIG. 1, the joint disorder risk evaluation device 300, the storage device 400, and the display device 500 are included in one pedestrian terminal 20. The pedestrian terminal 20 is, for example, a computer, a smart phone, a tablet, a head-mounted display such as smart glasses, a smart watch, or a smart band.

The joint disorder risk evaluation device 300, the storage device 400, and the display device 500 may not be included in one pedestrian terminal 20. For example, the joint disorder risk evaluation device 300 and the storage device 400 may be included in the cloud system and only the display device 500 may be included in the pedestrian terminal 20, which is a smartphone or the like.

The motion measurement device 100 has a function to measure a motion of the pedestrian 60. The motion in this exemplary embodiment refers to a motion such as a gait of the pedestrian 60.

For example, if the body of the pedestrian 60 is regarded as a rigid body link, the motion measurement device 100 measures information such as an angle and an angular velocity of each joint of the pedestrian 60, and a posture, a position, an acceleration, an angular velocity and the like of each body segment of the pedestrian 60.

Each joint refers to a joint that is measured by the motion measurement device 100. The joints to be evaluated for the joint disorder risk are any of joints. Once the joints to be evaluated for the joint disorder risk are determined, the joints to be measured are also determined.

For example, when a knee joint is an object of the evaluation of the joint disorder risk, the motion measurement device 100 measures motion of a knee joint and motion of an ankle joint. The reason why the motion measurement device 100 also measures motion of joints other than those to be evaluated is that, in general, a computation accuracy of the dynamics parameter described below will be higher when information of multiple joints is prepared. The motion measurement device 100 may measure motion of the hip joint.

A body segment is equivalent to a single mass of bones, such as a thigh, a lower thigh, a foot, a waist or a hip, a torso, a head or the like. The single mass of bones is a chunk that includes one bone and a surrounding area associated with the bone. The surrounding area is a part that exists within a range of movement with the bone as one rigid body without deformation.

The information measured by the motion measurement device 100 of this exemplary embodiment is not limited to the information described above. The motion measurement device 100 transmits the motion data, which is time-series data representing the measured motion of the pedestrian 60, to the joint disorder risk evaluation device 300. The motion measurement device 100 obtains the motion data which is time-series data representing the motion, by measuring the motion of the pedestrian 60.

FIG. 2 is an explanatory diagram showing an example of motion data of a knee joint. The motion data shown in FIG. 2 is obtained when a knee joint of a left leg is measured over one gait cycle which is a period from the time the left heel is grounded until the next left heel is grounded.

The motion data shown in FIG. 2 is in angle. That is, the closer the motion data value is to 0 degree, the more extended the knee joint is. Also, the closer the motion data value is to −90 degree, the more flexed the knee joint is.

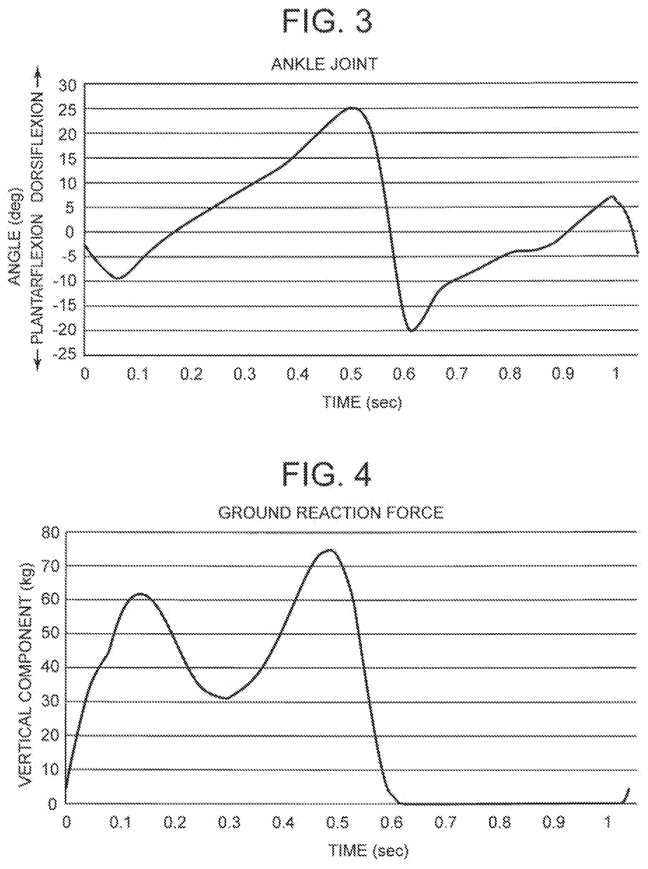

FIG. 3 is an explanatory diagram showing an example of motion data of an ankle joint. The motion data shown in FIG. 3 is obtained when an ankle joint of a left leg is measured over one gait cycle from the time the left heel is grounded until the next left heel is grounded.

The motion data shown in FIG. 3 is in angle. That is, the larger positive motion data value is, the more dorsiflexed the ankle joint is. In addition, the larger negative value of the motion data, the more the ankle joint is plantarflexed.

The motion measurement device 100 may measure a plurality of motions of the pedestrian 60. The motion measurement device 100 used in this exemplary embodiment is not limited to one.

The motion measurement device 100 is an IMU (Inertial Measurement Unit) having, for example, an accelerometer and an angular velocity meter. The IMU is attached to the thigh or shin, for example, using a band or the like. The IMU may be attached to both feet or only one foot.

It is preferred that the measurement range of the accelerometer included in the IMU includes the maximum acceleration at the attached position while the pedestrian 60 is walking. Similarly, it is preferred that the measurement range of the angular velocity meter included in the IMU includes the maximum angular velocity at the attached position while the pedestrian 60 is walking. The reason is that if the measurement range of the IMU does not correspond to the movement of the pedestrian 60, the accuracy of the computation of the dynamics parameter is reduced.

The motion measurement device 100 may be a smartphone having an accelerometer and an angular velocity meter. When motion of the knee joint is measured, for example, the IMU is attached below the knee and the smartphone is attached above the knee. That is, when a smartphone is used, instead of two IMUs for example, one IMU and one smartphone can perform the measurement. In other words, the smartphone may be used as the IMU.

If the motion of the ankle joint is measured, for example, the IMU is attached to the foot and below the knee of the pedestrian 60.

The motion measurement device 100 may be an optical motion capture device, a goniometer, a camera, or the like. The motion measurement device 100 of this exemplary embodiment is not limited to such examples.

The time interval for the motion measurement device 100 to measure the motion of the pedestrian 60 is not particularly limited. However, if the time interval of the measurement is too long, the computation accuracy of the dynamics parameter described below may decrease. In addition, if the time interval of the measurement is too short, the amount of transmitted motion data may be excessive.

Therefore, the motion measurement device 100 is preferable to measure the motion of the pedestrian 60 at 10 millisecond intervals, for example, taking into account a gait cycle of the pedestrian 60.

The ground reaction force measurement device 200 has a function of measuring ground reaction force applied to the pedestrian 60. The ground reaction force represents characteristics of force received by a plantar surface from a floor, such as three components of force (vertical component, lateral component, and fore-aft component) that comprise the force received by a plantar surface from the floor, a ground reaction force action point expressed as a coordinate value on the floor surface, and a rotational moment that represents the strength of the rotation of the force, etc.

The ground reaction force measurement device 200 transmits the ground reaction force data, which is time-series data representing the measured ground reaction force on the pedestrian 60, to the joint disorder risk evaluation device 300. The ground reaction force measurement device 200 obtains the ground reaction force data which is time-series data representing the ground reaction force, by measuring the ground reaction force applied to the pedestrian 60.

The ground reaction force measurement device 200 is, for example, a pressure gauge such as a strain gauge type pressure gauge, a capacitance type pressure gauge, or the like. The ground reaction force measurement device 200 may be a pressure gauge that measures the ground reaction force on the basis of a change in resistance value. The pressure gauge that measures the ground reaction force on the basis of a change in resistance value is attached under an insole (sock liner), for example.

It is preferred that the measurement range of the pressure gauge includes the maximum ground reaction force at the attached position while the pedestrian 60 is walking. The reason is that if a load beyond the measurement range is applied to the pressure gauge, such as at the point of landing during running, the accuracy of the computation of the dynamics parameter will decrease.

The ground reaction force measurement device 200 may be installed only on either a left left leg or a right left leg, or on both left legs. The ground reaction force measurement device 200 measures the ground reaction force applied to the pedestrian 60 at the portion where it is attached.

The ground reaction force measurement device 200 may be a force plate placed on the floor that is capable of measuring the ground reaction force applied to the pedestrian 60. The ground reaction force measurement device 200 of this exemplary embodiment is not limited to the examples described above.

The time interval at which the ground reaction force measurement device 200 measures the ground reaction force applied to the pedestrian 60 is not limited. However, if the time interval of the measurement is too long, the accuracy of the computation of the dynamics parameter described below may decrease. In addition, if the time interval of the measurement is too short, the amount of transmitted ground reaction force data may be excessive.

Therefore, the ground reaction force measurement device 200 is preferable to measure the ground reaction force applied to the pedestrian 60 at intervals of, for example, 10 milliseconds, taking into account a gait cycle of the pedestrian 60.

FIG. 4 is an explanatory diagram showing an example of the ground reaction force data. The ground reaction force data shown in FIG. 4 is ground reaction force data showing a vertical component partial of the ground reaction force applied to a left leg measured over one gait cycle which is a period from the time the left heel is grounded until the next left heel is grounded. The ground reaction force data shown in FIG. 4 is in kg.

The joint disorder risk evaluation device 300 receives the motion data from the motion measurement device 100 and the ground reaction force data from the ground reaction force measurement device 200, respectively. The joint disorder risk evaluation device 300 transmits a joint disorder risk indicator, which is an indicator representing the joint disorder risk determined using the received data, to the display device 500. The specific function and configuration of the joint disorder risk evaluation device 300 will be described separately with different drawings.

The memory device 400 has a function of storing predetermined data required to determine the joint disorder risk indicator of the pedestrian 60. The storage device 400 transmits the predetermined data required to determine the joint disorder risk indicator to the joint disorder risk evaluation device 300. The data stored in the storage device 400 will be described separately with different drawings.

The display device 500 has a function of displaying the joint disorder risk indicator received from the joint disorder risk evaluation device 300. The display device 500 may display at least one of the motion data and the ground reaction force data together with the joint disorder risk indicator.

(Example Configuration of the Joint Disorder Risk Evaluation Device 300)

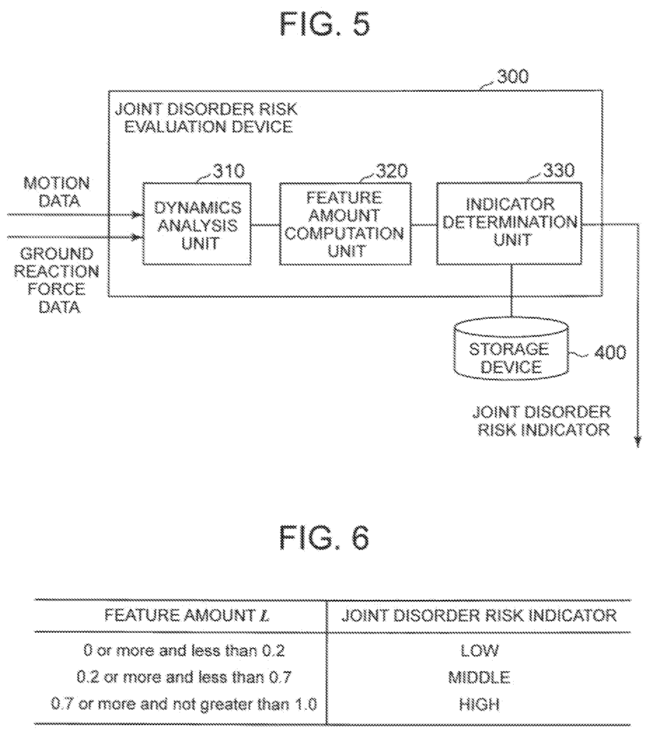

Next, referring to FIG. 5, the function and the configuration of the joint disorder risk evaluation device 300 included in the joint disorder risk evaluation system 10 of this exemplary embodiment will be described. FIG. 5 is a block diagram showing an example configuration of the joint disorder risk evaluation device 300 of the first exemplary embodiment.

As shown in FIG. 5, the joint disorder risk evaluation device 300 of this exemplary embodiment has a dynamics analysis unit 310, a feature amount computation unit 320, and an indicator determination unit 330. As shown in FIG. 5, the storage device 400 is communicatively connected to the indicator determination unit 330.

For convenience of explanation, a case will be taken where a joint to be evaluated is a knee joint as an example.

The dynamics analysis unit 310 has a function of computing a dynamics parameter at a joint to be evaluated. The dynamics parameter in this exemplary embodiment is a variable in an equation of motion representing motion of an object on which arbitrary force is acting. As the equation of motion, the equation of motion of a rigid body as well as the equation of motion of a material point is available.

The dynamics parameter is, for example, knee joint reaction force as described in formula (1), which is joint reaction force (the force acting between the distal end of a femur and the proximal end of the tibia) at the knee joint

[Math. 1]

\vec{JRFₖₙₑₑ}(t) Equation (1)

For example, the greater the knee joint reaction force, the stronger the knee is compressed.

The dynamics parameter may be a joint moment at the joint to be evaluated. Specific examples of joint moments are described, for example, in the patent literature 1.

In equation (1), t denotes the time (as in other formulas). For example, commonly known inverse dynamics computation is used to compute the knee joint reaction force shown in equation (1). When the inverse dynamics computation is used, the knee joint reaction force is computed as follows.

[Math. 2]

\vec{JRFₖₙₑₑ}(t)=m_{lowerthigh}\vec{α_{lowerthigh}}(t)−m_{lowerthigh}\vec{g}−\vec{JRFₐₙₖₗₑ}(t) Equation (2)

The m_{lowerthigh} in equation (2) denotes mass of a lower thigh. The first term on the right side of equation (2) denotes the product of mass of the lower thigh and a lower thigh acceleration. The lower thigh acceleration is expressed as follows.

[Math. 3]

\vec{α_{lowerthigh}}(t)=[α_{lowerthigh}_{ₓ}(t)α_{lowerthigh}_{_{y}}(t)α_{lowerthigh}_{_{z}}(t)]^{T}

The indices x, y, and z of each element indicate the lateral, fore-aft, and vertical directions, respectively (as in other formulas). The symbol T denotes the operation of the transposition (as in the other formulas). The second term on the right side of equation (2) denotes the product of mass of the lower leg and the gravitational acceleration. The third term on the right side of equation (2) denotes ankle joint reaction force. The ankle joint reaction force is computed as follows.

[Math. 4]

\vec{JRFₐₙₖₗₑ}(t)=m_{foot}\vec{α_{foot}}(t)−m_{foot}\vec{g}−\vec{GRF}(t) Equation (3)

The m_{foot} in equation (3) denotes mass of a foot. The first term on the right side of equation (3) denotes a product of mass of the foot part and foot acceleration. The foot acceleration is expressed as follows.

[Math. 5]

\vec{α_{foot}}(t)=[α_{foot}_{ₓ}(t)α_{foot}_{_{y}}(t)α_{foot}_{_{z}}(t)]^{T}

The second term on the right side of equation (3) denotes a product of mass of the foot and the gravitational acceleration. The third term on the right side of equation (3) denotes ground reaction force data. The ground reaction force data represents the ground reaction force, which is force that a plantar surface receives from the floor. The ground reaction force data is represented by a vector with three components of force (lateral component, fore-aft component and vertical component) as follows.

[Math. 6]

\vec{GRF}(t)=[GRFₓ(t)GRF_{y}(t)GRF_{z}(t)]^{T}

The motion data in equations (2)-(3) are the foot acceleration and the lower leg acceleration. That is, the dynamics analysis unit 310 computes the joint reaction force using the motion data and the ground reaction force data. The dynamics analysis unit 310 may estimate the ground reaction force data on the basis of the motion data by the following computation.

[Math. 7]

\vec{GRF}(t)=A·\vec{α_{foot}}(t)+B·\vec{α_{lowerthigh}}(t)+C·m+D Equation (4)

Note that A∈R^{3×3}, B∈R^{3×3}, C∈R^{3×1}, and D∈R^{3×1} in equation (4) represent the regression coefficients (R is a symbol for the entire set of real numbers), respectively. In addition, m in equation (4) represents weight of the pedestrian 60. That is, equation (4) is a linear regression equation with foot acceleration, lower leg acceleration, and body weight as explanatory variables. As described above, the dynamics analysis unit 310 may use the ground reaction force data estimated on the basis of the obtained motion data.

When the dynamics analysis unit 310 estimates ground reaction force data on the basis of motion data, the ground reaction force measurement device 200 may not be provided in the joint disorder risk evaluation system 10. It is also applicable to the second exemplary embodiment described below that the dynamics analysis unit 310 may estimate the ground reaction force data on the basis of the motion data and that the ground reaction force measurement device 200 may not be provided when the ground reaction force data is estimated.

The feature amount computation unit 320 has a function of computing a feature amount that represents a load repeatedly applied to a joint, on the basis of a dynamics parameter (e.g., joint reaction force) computed by the dynamics analysis unit 310.

The following is an example of feature amount computation. Magnitude of knee joint reaction force is represented by the equation (5).

[Math. 8]

JRFₖₙₑₑ(t)=|\vec{JRFₖₙₑₑ}(t)| Equation (5)

When a function, which is fourier-transformed magnitude of knee joint reaction force, is assumed X(f) (where f is the frequency), a power spectrum of X(f) is represented by |X(f)|². The power spectral density function Φ(f) is defined by the following equation, because the power spectrum is a function normalized in time.

$$\begin{matrix}[ Math . 9 ] & \\ Φ ( f ) = {lim}_{T \to ∞} \frac{1}{T} {\vert X ( f ) \vert}^{2} & Equation ( 6 )\end{matrix}$$

The feature amount L may be a value of the power spectral density function D(f) integrated over a predetermined frequency range (f_{H} to f_{L}[Hz]) as follows.

[Math. 10]

L=∫_{F}_{_{L}}^{f}^{_{H}}Φ(f)df Equation (7)

When the joint disorder risk of knee osteoarthritis is evaluated, it is desirable to set frequency components to f_{H} and f_{L} that is likely to cause degeneration of articular cartilage. For example, the non patent literature 1 describes that 60 instantaneous loads per minute cause degeneration of articular cartilage. Therefore, it is thought to set f_{H} to f_{L} to a frequency range around 1 Hz (for example, f_{L}=0.8, f_{H}=1.2).

The feature amount L in the equation (7) represents strength of knee joint reaction force that varies repeatedly by the frequency (period) in the frequency range f_{L} to f_{H}. The feature amount L is almost zero when the pedestrian 60 stands straight without moving. The reason is that since the joint reaction force during stand straight without moving is constant, the distribution of Φ(f) is concentrated at f=0.

When the pedestrian 60 is performing an action that repeatedly results in a load such as walking, the feature amount L is greater than 0. In other words, the feature amount L in the equation (7) is a feature amount in which the load repeatedly applied to the joint is taken into account because it is sensitive to the load repeatedly applied to the joint. The reason for considering the load repeatedly applied to the joint is because, as mentioned above, the load repeatedly applied to the joint is the main factor that increases the joint disorder risk.

The indicator determination unit 330 has a function of determining a joint disorder risk indicator on the basis of the feature amount L computed by the feature amount computation unit 320. In order to determine the joint disorder risk indicator, the indicator determination unit 330 refers, for example, to the joint disorder risk indicator table showing the correspondence between the feature value L and the joint disorder risk indicator. The joint disorder risk indicator table is information generated beforehand by a statistical or other method and stored in the storage device 400.

That is, in the present example, the storage device 400 stores a joint disorder risk indicator table indicating the correspondence between the feature L and the joint disorder risk indicator. The indicator determination unit 330 determines the joint disorder risk indicator using the stored joint disorder risk indicator table.

FIG. 6 is an explanatory diagram showing an example of a joint disorder risk indicator table of the first exemplary embodiment. As shown in FIG. 6, the joint disorder risk indicator table shows information wherein a predetermined range of feature amount L is associated to a joint disorder risk indicator. Specifically, the joint disorder risk indicator table indicates that the higher the value of the feature amount L is, the higher the joint disorder risk indicator is, and the lower the value of the feature amount L is, the lower the joint disorder risk indicator is.

The method of determining the joint disorder risk indicator is not limited to the method of referring to the joint disorder risk indicator table described above. For example, the indicator determination unit 330 may determine the joint disorder risk indicator by inputting a feature amount L into the decision model, which is a model for determining the joint disorder risk indicator generated in advance.

### [Description of Operation]

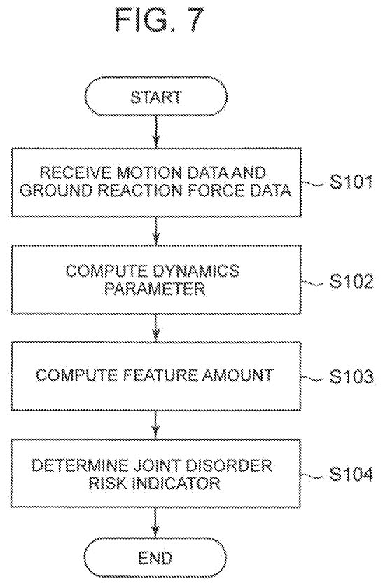

The operation of the joint disorder risk evaluation device 300 included in the joint disorder risk evaluation system 10 of this exemplary embodiment for evaluating joint disorder risk will be described below with reference to FIG. 7. FIG. 7 is a flowchart showing the operation of the joint disorder risk evaluation process by the joint disorder risk evaluation device 300 of the first exemplary embodiment.

First, the dynamics analysis unit 310 of the joint disorder risk evaluation device 300 receives the motion data transmitted from the motion measurement device 100 and the ground reaction force data transmitted from the ground reaction force measurement device 200 (step S101).

Next, the dynamics analysis unit 310 computes a dynamics parameter at the joint to be evaluated using the received motion data and the received ground reaction force data (step S102). Next, the dynamics analysis unit 310 inputs the computed the dynamics parameter to the feature amount computation unit 320.

Next, the feature amount computation unit 320 computes a feature amount representing a load repeatedly applied to the joint, using the dynamics parameter inputted from the dynamics analysis unit 310 (step S103). Then, the feature amount computation unit 320 inputs the computed feature amount to the indicator determination unit 330.

Next, the indicator determination unit 330 determines the joint disorder risk indicator using the feature amount inputted from the feature amount computation unit 320 (step S104). Next, the indicator determination unit 330 outputs the determined joint disorder risk indicator. After outputting the determined joint disorder risk indicator, the joint disorder risk evaluation device 300 terminates the joint disorder risk evaluation process.

### [Description of Effect]

The joint disorder risk evaluation device 300 of the joint disorder risk evaluation system 10 of this exemplary embodiment can evaluate the joint disorder risk of the pedestrian 60 by executing the joint disorder risk evaluation process shown in FIG. 7.

A user using the joint disorder risk evaluation device 300 of this exemplary embodiment can evaluate the joint disorder risk with higher accuracy. The reason is that the feature amount computation unit 320 of the joint disorder risk evaluation device 300 computes a feature amount that takes into account the load repeatedly applied to the joint, and the indicator determination unit 330 uses the computed feature amount to determine the joint disorder risk indicator.

The joint disorder risk evaluation device 300 of this exemplary embodiment can also evaluate joints other than the knee joint. For example, the joint disorder risk evaluation device 300 may evaluate the joint disorder risk of a joint of a lumbar vertebrae of a person who routinely carries heavy objects, such as a care worker or a carrier. When the joint disorder risk of the joint of the lumbar vertebrae is evaluated, force applied to the upper body of the object person is measured.

The joint disorder risk evaluation device 300 of this exemplary embodiment may evaluate the joint disorder risk of a joint of a robot rather than a person. In particular, the joint disorder risk evaluation device 300 may evaluate the joint disorder risk of a joint of an automobile assembly robot, a life support robot or the like.

### Exemplary Embodiment 2

Next, a second exemplary embodiment of the presymptomatic locomotive syndrome countermeasure system according to the present invention will be described with reference to the drawings. The presymptomatic locomotive syndrome countermeasure system of this exemplary embodiment is a system which is an application of the joint disorder risk evaluation system 10 of the first exemplary embodiment.

Locomotive syndrome (musculoskeletal deterioration) is a condition in which mobility is impaired due to musculoskeletal disorders. Patients with locomotive syndrome often have limited activities of daily living, such as not being able to go shopping, climb stairs, or make it difficult to engage in group activities due to slower walk as compared with normal individuals. When activities of daily living are limited, quality of life (QoL) of the patient may be reduced, because the range of activities of the patient can perform is less than it was before they fell into locomotive syndrome.

In addition, limitations in activities of daily living may increase the risk of requiring assistance or requiring nursing care. In other words, as the number of patients suffering from locomotive syndrome increases, social security costs are expected to increase.

Typical cases of locomotive syndrome include knee osteoarthritis and lumbar spondylosis. Knee osteoarthritis and lumbar spondylosis are symptoms of wear and tear on loaded articular cartilage, which causes inflammation of the joints and pain in the knees and hips.

In order to prevent the appearance of knee osteoarthritis and lumbar spondylosis, it is important not to increase the load on articular cartilage in daily life. If the load on articular cartilage is not increased, the onset of locomotive syndrome will be prevented. Alternatively, the onset of locomotive syndrome can be delayed.

When the onset of locomotive syndrome is prevented or delayed, healthy expectancy of the patient is extended. Measures that reduce the load placed on the articular cartilage before the onset of locomotive syndrome are referred to as presymptomatic disease countermeasure.

However, when developed locomotive syndrome is mild, it is difficult for the patient to recognize the onset of locomotive syndrome. In addition, even if the patient is aware of the onset of locomotive syndrome, the patient may believe that it is not enough to warrant a visit to the hospital, and then the patient comes to the hospital when the condition has become severe enough to interfere with their daily lives. For these reasons, it is difficult to take countermeasures to presymptomatic disease of locomotive syndrome.

To the above problem, the user as the pedestrian 60 can grasp the joint disorder risk at the presymptomatic disease stage using the joint disorder risk evaluation device 300 of the first exemplary embodiment. However, since the user does not have specialized knowledge in general, the user has a problem that the user does not know what specific countermeasures should be taken to prevent joint disorders even if the user grasps the joint disorder risk.

In the following description, knee osteoarthritis, which is one of the typical cases of locomotive syndrome, is described as an evaluation object of joint disorder risk. The following description also assumes that a measurement sensor of the motion measurement device 100 and a measurement sensor of the ground reaction force measurement device 200 are attached to the leg (especially the thigh, the lower leg, and the foot).

The evaluation object of the presymptomatic locomotive syndrome countermeasure system of this exemplary embodiment is not limited to knee osteoarthritis. The object for evaluation may be, for example, osteoarthritis of the hip or low back pain. Furthermore, the object for evaluation may be neck pain, stiff shoulder or the like, other than locomotive syndrome.

When symptoms other than knee osteoarthritis are to be evaluated, the measurement sensors will be appropriately placed at a location where the joint reaction force at the joint to be evaluated can be measured.

### [Explanation of Configuration]

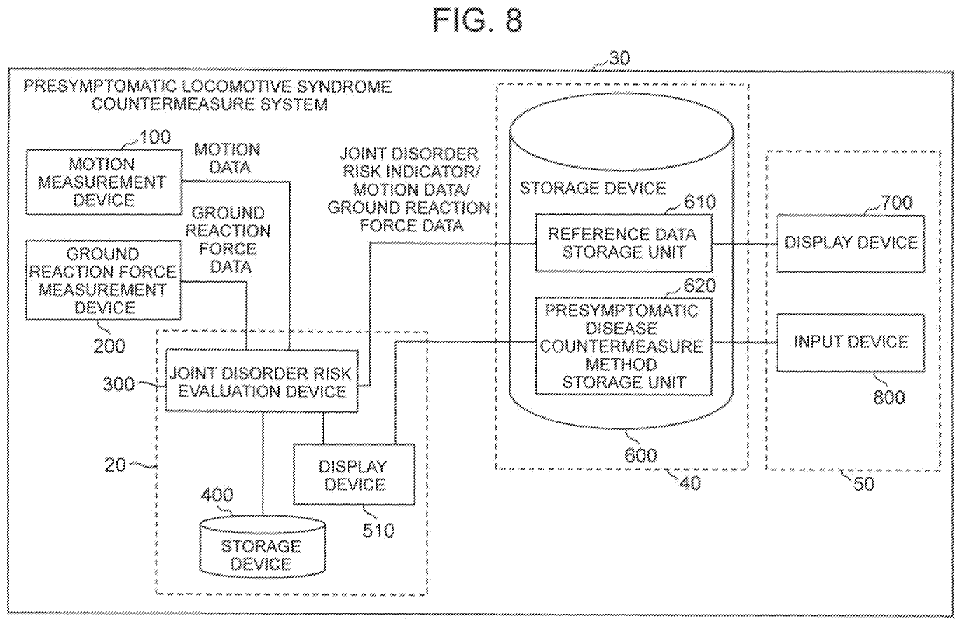

FIG. 8 is a block diagram showing an example configuration of the second exemplary embodiment of a presymptomatic locomotive syndrome (musculoskeletal deterioration) countermeasure system according to the present invention. As shown in FIG. 8, the presymptomatic locomotive syndrome countermeasure system 30 includes a motion measurement device 100, a ground reaction force measurement device 200, a joint disorder risk evaluation device 300, a storage device 400, a display device 510, a storage device 600, a display device 700, and an input device 800.

The motion measurement device 100, the ground reaction force measurement device 200, the joint disorder risk evaluation device 300, and the storage device 400 of this exemplary embodiment are components that are also used in the joint disorder risk evaluation system 10 of the first exemplary embodiment.

As shown in FIG. 8, the joint disorder risk evaluation device 300, the storage device 400, and the display device 510 are included in one pedestrian terminal 20, as in the first exemplary embodiment. The storage device 600 is also included in the server 40.

As shown in FIG. 8, the display device 700 and the input device 800 are included in one inputter terminal 50. The joint disorder risk evaluation device 300 and the storage device 400 may be included in the server 40 instead of the pedestrian terminal 20.

The storage device 600 includes a reference data storage unit 610 and a presymptomatic disease countermeasure method storage unit 620. The reference data storage unit 610 is inputted with the obtained motion data, the obtained ground reaction force data, and the determined joint disorder risk indicator from the joint disorder risk evaluation device 300.

The reference data storage unit 610 has a function of storing each inputted data as reference data. The reference data storage unit 610 transmits the stored reference data to the display device 700.

Presymptomatic disease countermeasure method data, which is data indicating a presymptomatic disease countermeasure method described below, is inputted to the presymptomatic disease countermeasure method storage unit 620 from the input device 800. The presymptomatic disease countermeasure method storage unit 620 has a function of storing the inputted presymptomatic disease countermeasure method data. The presymptomatic disease countermeasure method storage unit 620 transmits the stored presymptomatic disease countermeasure method data to the display device 510.

The storage device 600 stores correspondence between the pedestrian 60, the reference data stored in the reference data storage unit 610 and the presymptomatic disease countermeasure method data stored in the presymptomatic disease countermeasure method storage unit 620.

In addition to the function of the display device 500 of the joint disorder risk evaluation system 10, the display device 510 has a function of displaying the presymptomatic disease countermeasure method data received from the presymptomatic disease countermeasure method storage unit 620.

The display device 700 has a function of displaying the reference data received from the reference data storage unit 610.

The input device 800 has an interface that is used, for example, to input the presymptomatic disease countermeasure method. The presymptomatic disease countermeasure method is a specific method for reducing the joint disorder risk. The presymptomatic disease countermeasure method is presentation of a strength training plan, recommending use of cushioned shoes, or recommending that people refrain from carrying heavy objects, for example.

In addition, if it is better for a medical institution to take countermeasures directly for reasons such as difficulty of the pedestrian 60 by oneself in carrying out voluntary countermeasures, the presymptomatic disease countermeasure method will be to recommend that he or she visits a medical institution.

The countermeasures for preventing development of symptoms are inputted to the input device 800. The presymptomatic disease countermeasure method data can be text data, audio data, image data, etc. The format of the presymptomatic disease countermeasure method data can be in any format that can be used in the presymptomatic locomotive syndrome countermeasure system 30.

Therefore, the display device 510 of this exemplary embodiment displays the joint disorder risk indicator determined by the indicator determination unit 330 together with the countermeasures to prevent the development of symptoms caused by the joint reaction force computed by the dynamics analysis unit 310.

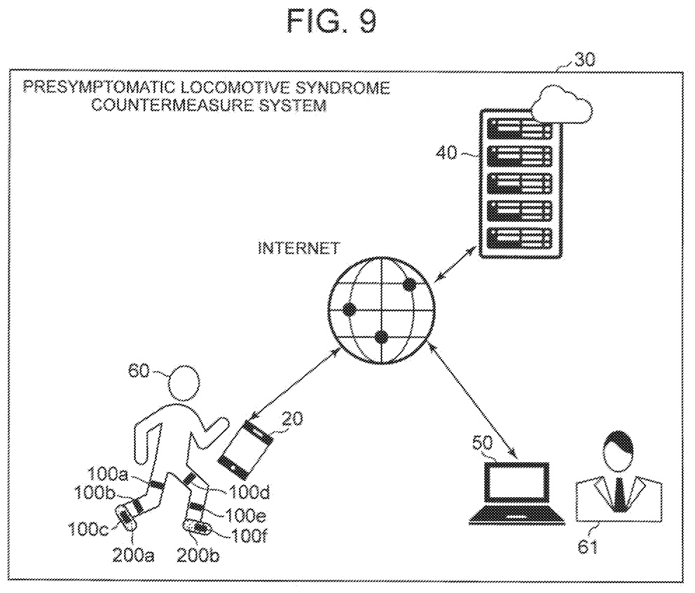

FIG. 9 is an explanatory diagram showing an example of the second exemplary embodiment of the presymptomatic locomotive syndrome countermeasure system 30. The presymptomatic locomotive syndrome countermeasure system 30 shown in FIG. 9 includes a pedestrian terminal 20, a server 40, an inputter terminal 50, motion measurement devices 100a-100f, and ground reaction force measurement devices 200a-200b.

As shown in FIG. 9, the motion measurement device 100a is attached to the left thigh of the pedestrian 60. The motion measurement device 100b is attached to the left lower thigh of the pedestrian 60. The motion measurement device 100c is attached to the left foot of the pedestrian 60.

Further, as shown in FIG. 9, the motion measurement device 100d is attached to the right thigh of the pedestrian 60. The motion measurement device 100e is attached to the right lower leg of the pedestrian 60. The motion measurement device 100f is attached to the right foot of the pedestrian 60. The respective motion measurement devices 100a to 100f perform the same motion as the motion measurement device 100 of the joint disorder risk evaluation system 10 does.

Further, as shown in FIG. 9, the ground reaction force measurement device 200a is attached to the left plantar of the pedestrian 60. The ground reaction force measurement device 200b is attached to the right plantar of the pedestrian 60. The respective ground reaction force measurement device 200a and the ground reaction force measurement device 200b perform the same action as the ground reaction force measurement device 200 of the joint disorder risk evaluation system 10 does.

The inputter terminal 50 displays the reference data sent to the display or the like. The inputter 61, who enters the presymptomatic disease countermeasure method into the inputter terminal 50, enters the presymptomatic disease countermeasure method into the inputter terminal 50 through an interface such as a keyboard or a touch panel.

The inputter 61 determines contents of the inputted presymptomatic disease countermeasure method. The inputter 61 is preferably an expert having knowledge of a joint disorder or locomotive syndrome, such as a physician, a physical therapist or the like. The inputter terminal 50 transmits to the server 40 the presymptomatic disease countermeasure method data indicating the presymptomatic disease countermeasure method inputted.

The communication means between the pedestrian terminal 20, the server 40, and the inputter terminal 50 are not limited. If the presymptomatic locomotive syndrome countermeasure system 30 is required to be a convenient system that enables the pedestrian 60 to receive the presymptomatic disease countermeasure method from the inputter terminal 50 located in a remote location, the communication means between the pedestrian terminal 20 and the internet is preferably a wireless communication means.

### [Description of Operation]

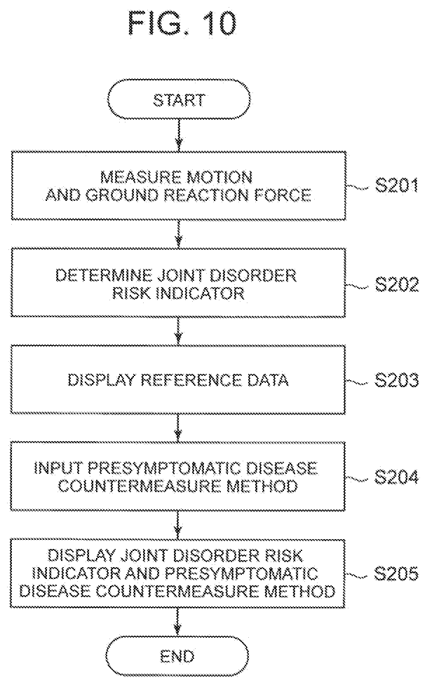

Hereinafter, the operation of displaying the presymptomatic disease countermeasure method by the presymptomatic locomotive syndrome countermeasure system 30 of this exemplary embodiment will be described with reference to FIG. 10. FIG. 10 is a flowchart showing an operation of a presymptomatic disease countermeasure method displaying process by the presymptomatic locomotive syndrome countermeasure system 30 of the second exemplary embodiment.

First, the motion measurement device 100 of the presymptomatic locomotive syndrome countermeasure system 30 measures motion of the pedestrian 60. In addition, the ground reaction force measurement device 200 of the presymptomatic locomotive syndrome countermeasure system 30 measures ground reaction force applied to the pedestrian 60 (step S201).

The motion measurement device 100 then transmits the obtained motion data to the joint disorder risk evaluation device 300 and the reference data storage unit 610 of the storage device 600. The ground reaction force measurement device 200 transmits the obtained ground reaction force data to the joint disorder risk evaluation device 300 and the reference data storage unit 610 of the storage device 600.

Next, the joint disorder risk evaluation device 300 determines the joint disorder risk indicator using the motion data transmitted from the motion measurement device 100 and the ground reaction force data transmitted from the ground reaction force measurement device 200 (step S202). The process of step S202 corresponds to the processes of steps S101 to S104 in the first exemplary embodiment.

The joint disorder risk evaluation device 300 then transmits the data indicating the determined joint disorder risk indicator to the display device 510 and the reference data storage unit 610 in the storage device 600.

The display device 700 then displays the reference data stored in the reference data storage unit 610 in the storage device 600 (step S203).

Next, the inputter 61 inputs the presymptomatic disease countermeasure method to the input device 800 (step S204). The input device 800 transmits the presymptomatic disease countermeasure method data indicating the presymptomatic disease countermeasure method inputted to the presymptomatic disease countermeasure method storage unit 620 in the storage device 600.

Next, the display device 510 receives data indicating the joint disorder risk indicator from the joint disorder risk evaluation device 300. The display device 510 also receives the presymptomatic disease countermeasure method data from the presymptomatic disease countermeasure method storage unit 620 in the storage device 600.

Next, the display device 510 displays the received data indicating the joint disorder risk indicator and the received presymptomatic disease countermeasure method data to the pedestrian 60 (step S205). After displaying the data, the presymptomatic locomotive syndrome countermeasure system 30 terminates the process of displaying the presymptomatic disease countermeasure method.

### [Description of Effect]

The display device 510 of the presymptomatic locomotive syndrome countermeasure system 30 of this exemplary embodiment can simultaneously present to the user a joint disorder risk indicator determined with accuracy by the joint disorder risk evaluation device 300 and a presymptomatic disease countermeasure method.

In other words, when the presymptomatic locomotive syndrome countermeasure system 30 is used, an ordinary user who is not aware of the onset of locomotive syndrome, or an ordinary user who is aware of the onset of locomotive syndrome but does not know what countermeasures to take, can take effective presymptomatic disease countermeasures.

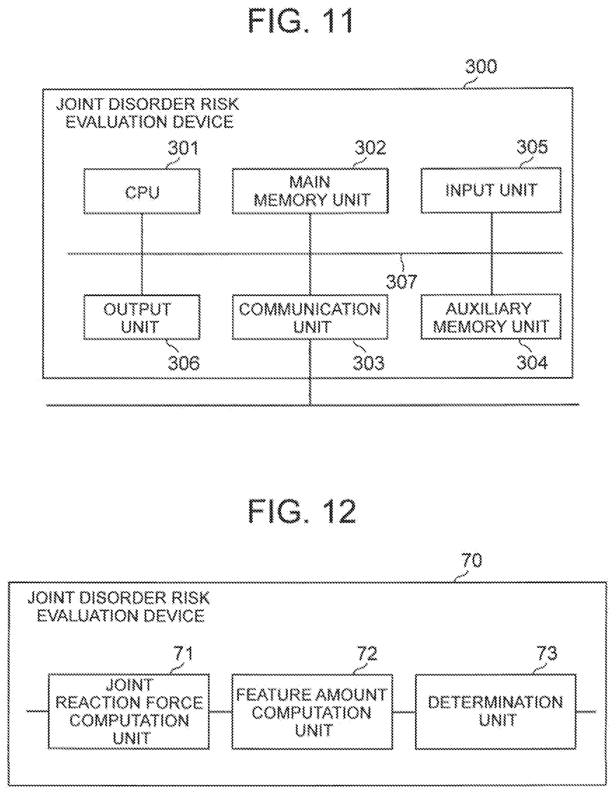

Specific examples of a hardware configuration of the joint disorder risk evaluation device 300 in each exemplary embodiment will be described below. FIG. 11 is an explanatory diagram showing an example hardware configuration of the joint disorder risk evaluation device 300 according to the present invention.

The joint disorder risk evaluation device 300 shown in FIG. 11 is implemented by a CPU (Central Processing Unit) 301, a main memory unit 302, a communication unit 303, and an auxiliary memory unit 304. It may also be provided with an input unit 305 for a user to operate and an output unit 306 for presenting a processing result or a progress of the processing contents to the user.

It is noted that the joint disorder risk evaluation device 300 shown in FIG. 11 may be implemented by a DSP (Digital Signal Processor) instead of the CPU 301. Alternatively, the joint disorder risk evaluation device 300 shown in FIG. 11 may be implemented by with both the CPU 301 and the DSP.

The main memory unit 302 is used as a working area for data and a temporary storage area for data. The main memory unit 302 is, for example, a RAM (Random Access Memory).

The communication unit 303 has a function of inputting and outputting data to and from peripheral devices through a wired network or a wireless network (information communication network).

The auxiliary memory unit 304 is a non-transitory tangible storage medium. Non-transitory tangible storage media include, for example, a magnetic disk, an optical magnetic disk, a CD-ROM (Compact Disk Read Only Memory), a DVD-ROM (Digital Versatile Disk Read Only Memory) and a semiconductor memory.

The input unit 305 has a function to input data and processing instructions. The input unit 305 is an input device, such as a keyboard, a mouse, and the like, for example.

The output unit 306 has a function to output data. The output unit 306 is a display device such as a liquid crystal display device or the like, or a printing device such as a printer or the like.

As shown in FIG. 11, in the joint disorder risk evaluation device 300, each component is connected to a system bus 307.

The auxiliary memory unit 304 stores programs for realizing, for example, the dynamics analysis unit 310, the feature amount computation unit 320, and the indicator determination unit 330. The dynamics analysis unit 310 and the indicator determination unit 330 may also execute communication processes through the communication unit 303.

The joint disorder risk evaluation device 300 may be realized by hardware. For example, the joint disorder risk evaluation device 300 may be implemented with a circuit that includes hardware components such as LSI (Large Scale Integration) with programs inside that realize the functions shown in FIG. 5.

The joint disorder risk evaluation device 300 may be realized by software by executing a program in which the CPU 301 shown in FIG. 11 provides the functions that each component has.

When realized by the software, each function is realized by the CPU 301 loading a program stored in the auxiliary memory unit 304 into the main memory unit 302 and executing the program to control the operation of the joint disorder risk evaluation device 300.

Some or all of components may be realized by a general purpose circuit (circuitry) or a dedicated circuit, processors, etc., or a combination thereof. These may be configured by a single chip or by a plurality of chips connected through a bus. Some or all of components may be realized by a combination of the above described circuits and the like with a program.

When a part or all of components are realized by a plurality of information processing devices, circuits, or the like, the plurality of information processing devices, circuits, or the like may be centrally located or distributed. For example, the information processing devices, circuits, or the like may be realized as a client server system, a cloud computing system, or the like, each of which is connected through a communication network.

Next, an outline of the present invention will be described. FIG. 12 is a block diagram showing an outline of the joint disorder risk evaluation device according to the present invention. The joint disorder risk evaluation device 70 according to the present invention comprises a joint reaction force computation unit 71 (for example, the dynamics analysis unit 310) which computes joint reaction force at a joint to be evaluated among joints of an object, using motion data that is time-series data representing motion of the object, and ground reaction force data that is time-series data representing ground reaction force applied to the object, a feature amount computation unit 72 (for example, the feature amount computation unit 320) which computes a feature amount representing a load repeatedly applied to the joint to be evaluated on the basis of the computed joint reaction force, and a determination unit 73 (for example, the indicator determination unit 330) which determines a joint disorder risk indicator that is an indicator representing a joint disorder risk that is the risk of causing joint disorder, on the basis of the computed feature amount.

With such a configuration, the joint disorder risk evaluation device can evaluate the joint disorder risk with higher accuracy.

In addition, the joint reaction force computation unit 71 may use the motion data obtained from motion measurement means which measures the motion of the object. Further, the joint reaction force computation unit 71 may use the ground reaction force data obtained from ground reaction force measurement means which measures the ground reaction force applied to the object.

With such a configuration, the joint disorder risk evaluation device can evaluate the joint disorder risk with higher accuracy.

The joint reaction force computation unit 71 may also estimate the ground reaction force data on the basis of the obtained motion data and use the estimated ground reaction force data.

With such a configuration, the joint disorder risk evaluation device can evaluate the joint disorder risk if motion data is obtained.

The determination unit 73 may also determine the joint disorder risk indicator using information indicating correspondence between the feature amount and the joint disorder risk indicator.

With such a configuration, the joint disorder risk evaluation device can evaluate the joint disorder risk on the basis of the correspondence between the feature amount indicated by previously obtained data and the joint disorder risk indicator.

The joint reaction force computation unit 71 may compute a joint moment at the joint to be evaluated, and the feature amount computation unit 72 may compute the feature amount on the basis of the computed joint moment.

With such a configuration, the joint disorder risk evaluation device can evaluate the joint disorder risk using a joint moment.

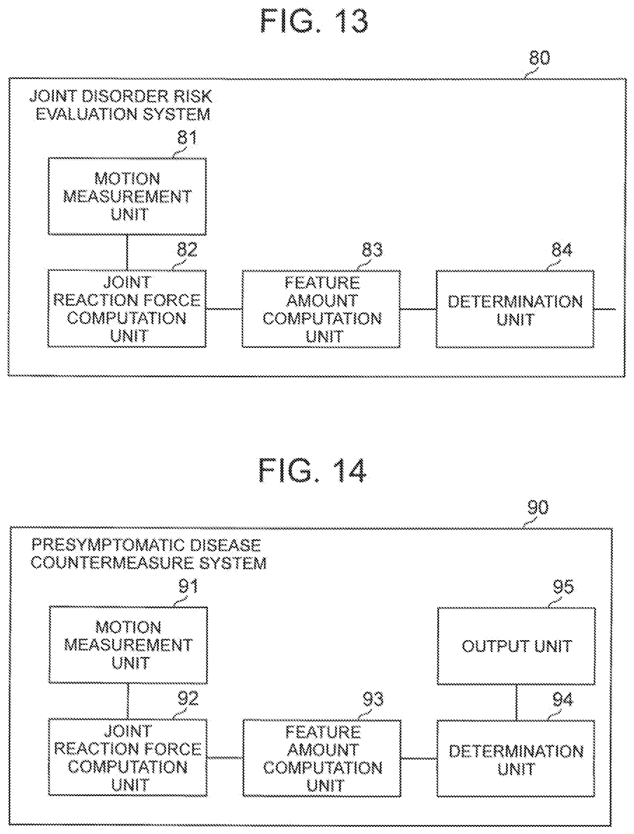

FIG. 13 is a block diagram showing an outline of the joint disorder risk evaluation system according to the present invention. The joint disorder risk evaluation system 80 according to the present invention comprises a motion measurement unit 81 (for example, the motion measurement device 100) which obtains motion data that is time-series data representing motion, by measuring the motion of an object, a joint reaction force computation unit 82 (for example, dynamics analysis unit 310) which computes joint reaction force at a joint to be evaluated among joints of the object, using the obtained motion data and ground reaction force data that is time-series data representing ground reaction force applied to the object, a feature amount computation unit 83 (for example, the feature amount computation unit 320) which computes a feature amount representing a load repeatedly applied to the joint to be evaluated on the basis of the computed joint reaction force, and a determination unit 84 (for example, the indicator determination unit 330) which determines a joint disorder risk indicator that is an indicator representing a joint disorder risk that is the risk of causing joint disorder, on the basis of the computed feature amount.

With such a configuration, the joint disorder risk evaluation system can evaluate the joint disorder risk with higher accuracy.

The joint disorder risk evaluation system 80 comprises a ground reaction force measurement unit (for example, the ground reaction force measurement device 200) which obtains ground reaction force data representing the ground reaction force by measuring the ground reaction force applied to the object, wherein the joint reaction force computation unit 82 uses the obtained ground reaction force data.

With such a configuration, the joint disorder risk evaluation system can evaluate the joint disorder risk with higher accuracy.

The joint reaction force computation unit 82 may also estimate the ground reaction force data on the basis of the obtained motion data and use the estimated ground reaction force data.

With such a configuration, the joint disorder risk evaluation system can evaluate the joint disorder risk even if the ground reaction force measurement unit is not provided.

The joint disorder risk evaluation system 80 comprises a storage unit (for example, storage device 400) which stores information indicating correspondence between the feature amount and the joint disorder risk indicator, wherein the determination unit 84 may use the stored information to determine the joint disorder risk indicator.

With such a configuration, the joint disorder risk evaluation system can evaluate the joint disorder risk on the basis of the correspondence between the feature amount indicated by previously obtained data and the joint disorder risk indicator.

The joint reaction force computation unit 82 may compute a joint moment at the joint to be evaluated, and the feature amount computation unit 83 may compute the feature amount on the basis of the computed joint moment.

With such a configuration, the joint disorder risk evaluation system can evaluate the joint disorder risk using a joint moment.

FIG. 14 is a block diagram showing an outline of the presymptomatic disease countermeasure system. The presymptomatic disease countermeasure system 90 according to the present invention comprises a motion measurement unit 91 (for example, the motion measurement device 100) which obtains motion data that is time-series data representing motion, by measuring the motion of an object, a joint reaction force computation unit 92 (for example, the dynamics analysis unit 310) which computes joint reaction force at a joint to be evaluated among joints of the object, using the obtained motion data and ground reaction force data that is time-series data representing ground reaction force applied to the object, a feature amount computation unit 93 (for example, the feature amount computation unit 320) which computes a feature amount representing a load repeatedly applied to the joint to be evaluated on the basis of the computed joint reaction force, a determination unit 94 (for example, the indicator determination unit 330) which determines a joint disorder risk indicator that is an indicator representing a joint disorder risk that is the risk of causing joint disorder, on the basis of the computed feature amount, and an output unit 95 (for example, the display device 510) which outputs the determined joint disorder risk indicator together with countermeasures for preventing development of symptoms caused by the computed joint reaction force.

With such a configuration, the presymptomatic disease countermeasure system can evaluate the joint disorder risk with higher accuracy.

The presymptomatic disease countermeasure system 90 may also comprise a first storage unit (for example, the reference data storage unit 610) which stores the motion data, the ground reaction force data, and a joint disorder risk indicator as reference data, and a display unit (for example, the display device 700) which displays the stored reference data.

With such a configuration, the presymptomatic disease countermeasure system can present the joint disorder risk indicator to experts.

The presymptomatic disease countermeasure system 90 may also comprise an input unit (for example, the input device 800) to which the countermeasures for preventing development of symptoms are inputted.

With such a configuration, the presymptomatic disease countermeasure system can use the presymptomatic disease countermeasure method entered by an expert according to the displayed reference data.

The presymptomatic disease countermeasure system 90 comprises a ground reaction force measurement unit (for example, the ground reaction force measurement device 200) which obtains the ground reaction force data representing the ground reaction force by measuring the ground reaction force applied to the object, wherein the joint reaction force computation unit 92 may use the obtained ground reaction force data.

With such a configuration, the presymptomatic disease countermeasure system can evaluate the joint disorder risk with higher accuracy.

The joint reaction force computation unit 92 may also estimate the ground reaction force data on the basis of the obtained motion data and use the estimated ground reaction force data.

With such a configuration, the presymptomatic disease countermeasure system can evaluate the joint disorder risk even if the ground reaction force measurement unit is not provided.

The presymptomatic disease countermeasure system 90 comprises a second storage unit (for example, the storage device 400) which stores information indicating correspondence between the feature amount and the joint disorder risk indicator, wherein the determination unit 94 may use the stored information to determine the joint disorder risk indicator.

With such a configuration, the presymptomatic disease countermeasure system can evaluate the joint disorder risk on the basis of the correspondence between the feature amount indicated by previously obtained data and the joint disorder risk indicator.

The joint reaction force computation unit 92 may compute a joint moment at the joint to be evaluated, and the feature amount computation unit 93 may compute the feature amount on the basis of the computed joint moment.

With such a configuration, the presymptomatic disease countermeasure system can evaluate the joint disorder risk using a joint moment.

While the present invention has been described with reference to the exemplary embodiments and examples, the present invention is not limited to the aforementioned exemplary embodiments and examples. Various changes understandable to those skilled in the art within the scope of the present invention can be made to the structures and details of the present invention.

The aforementioned exemplary embodiments can be described as supplementary notes mentioned below, but are not limited to the following supplementary notes.

(Supplementary note 1) A joint disorder risk evaluation device comprising: a joint reaction force computation unit which computes joint reaction force at a joint to be evaluated among joints of an object, using motion data that is time-series data representing motion of the object, and ground reaction force data that is time-series data representing ground reaction force applied to the object, a feature amount computation unit which computes a feature amount representing a load repeatedly applied to the joint to be evaluated on the basis of the computed joint reaction force, and a determination unit which determines a joint disorder risk indicator that is an indicator representing a joint disorder risk that is the risk of causing joint disorder, on the basis of the computed feature amount.

(Supplementary note 2) The joint disorder risk evaluation device according to supplementary note 1, wherein the joint reaction force computation unit uses the motion data obtained from motion measurement means which measures the motion of the object.

(Supplementary note 3) The joint disorder risk evaluation device according to supplementary note 1 or 2, wherein the joint reaction force computation unit uses the ground reaction force data obtained from ground reaction force measurement means which measures the ground reaction force applied to the object.

(Supplementary note 4) The joint disorder risk evaluation device according to supplementary note 2, wherein the joint reaction force computation unit estimates the ground reaction force data on the basis of the obtained motion data, and uses the estimated ground reaction force data.

(Supplementary note 5) The joint disorder risk evaluation device according to any one of supplementary notes 1 to 4, wherein the determination unit determines the joint disorder risk indicator using information indicating correspondence between the feature amount and the joint disorder risk indicator.

(Supplementary note 6) The joint disorder risk evaluation device according to any one of supplementary notes 1 to 5, wherein the joint reaction force computation unit computes a joint moment at the joint to be evaluated and, the feature amount computation unit computes the feature amount on the basis of the computed joint moment.

(Supplementary note 7) A joint disorder risk evaluation system comprising: a motion measurement unit which obtains motion data that is time-series data representing motion, by measuring the motion of an object, a joint reaction force computation unit which computes joint reaction force at a joint to be evaluated among joints of the object, using the obtained motion data and ground reaction force data that is time-series data representing ground reaction force applied to the object, a feature amount computation unit which computes a feature amount representing a load repeatedly applied to the joint to be evaluated on the basis of the computed joint reaction force, and a determination unit which determines a joint disorder risk indicator that is an indicator representing a joint disorder risk that is the risk of causing joint disorder, on the basis of the computed feature amount.

(Supplementary note 8) The joint disorder risk evaluation system according to supplementary note 7, further comprising a ground reaction force measurement unit which obtains ground reaction force data representing the ground reaction force by measuring the ground reaction force applied to the object, wherein the joint reaction force computation unit uses the obtained ground reaction force data.

(Supplementary note 9) The joint disorder risk evaluation system according to supplementary note 7, wherein the joint reaction force computation unit estimates the ground reaction force data on the basis of the obtained motion data, and uses the estimated ground reaction force data.

(Supplementary note 10) The joint disorder risk evaluation system according to any one of supplementary notes 7 to 9, further comprising a storage unit which stores information indicating correspondence between the feature amount and the joint disorder risk indicator, wherein the determination unit uses the stored information to determine the joint disorder risk indicator.

(Supplementary note 11) The joint disorder risk evaluation system according to any one of supplementary notes 7 to 10, wherein the joint reaction force computation unit computes a joint moment at the joint to be evaluated and, the feature amount computation unit computes the feature amount on the basis of the computed joint moment.

(Supplementary note 12) A presymptomatic disease countermeasure system comprising: a motion measurement unit which obtains motion data that is time-series data representing motion, by measuring the motion of an object, a joint reaction force computation unit which computes joint reaction force at a joint to be evaluated among joints of the object, using the obtained motion data and ground reaction force data that is time-series data representing ground reaction force applied to the object, a feature amount computation unit which computes a feature amount representing a load repeatedly applied to the joint to be evaluated on the basis of the computed joint reaction force, a determination unit which determines a joint disorder risk indicator that is an indicator representing a joint disorder risk that is the risk of causing joint disorder, on the basis of the computed feature amount, and an output unit which outputs the determined joint disorder risk indicator together with countermeasures for preventing development of symptoms caused by the computed joint reaction force.

(Supplementary note 13) The presymptomatic disease countermeasure system according to supplementary note 12, further comprising: a first storage which stores the motion data, the ground reaction force data, and a joint disorder risk indicator as reference data, and a display unit which displays the stored reference data.

(Supplementary note 14) The presymptomatic disease countermeasure system according to supplementary note 12 or 13, further comprising an input unit to which the countermeasures for preventing development of symptoms are inputted.

(Supplementary note 15) The presymptomatic disease countermeasure system according to any one of supplementary notes 12 to 14, further comprising a ground reaction force measurement unit which obtains the ground reaction force data representing the ground reaction force by measuring the ground reaction force applied to the object, wherein the joint reaction force computation unit uses the obtained ground reaction force data.

(Supplementary note 16) The presymptomatic disease countermeasure system according to any one of supplementary notes 12 to 14, wherein the joint reaction force computation unit estimates the ground reaction force data on the basis of the obtained motion data, and uses the estimated ground reaction force data.

(Supplementary note 17) The presymptomatic disease countermeasure system according to any one of supplementary notes 12 to 16, further comprising a second storage unit which stores information indicating correspondence between the feature amount and the joint disorder risk indicator, wherein the determination unit uses the stored information to determine the joint disorder risk indicator.

(Supplementary note 18) The presymptomatic disease countermeasure system according to any one of supplementary notes 12 to 17, wherein the joint reaction force computation unit computes a joint moment at the joint to be evaluated and, the feature amount computation unit computes the feature amount on the basis of the computed joint moment.

(Supplementary note 19) A joint disorder risk evaluation method comprising: computing joint reaction force at a joint to be evaluated among joints of an object, using motion data that is time-series data representing motion of the object, and ground reaction force data that is time-series data representing ground reaction force applied to the object, computing a feature amount representing a load repeatedly applied to the joint to be evaluated on the basis of the computed joint reaction force, and determining a joint disorder risk indicator that is an indicator representing a joint disorder risk that is the risk of causing joint disorder, on the basis of the computed feature amount.

(Supplementary note 20) A joint disorder risk evaluation method comprising: obtaining motion data that is time-series data representing motion, by measuring the motion of an object, computing joint reaction force at a joint to be evaluated among joints of the object, using the obtained motion data and ground reaction force data that is time-series data representing ground reaction force applied to the object, computing a feature amount representing a load repeatedly applied to the joint to be evaluated on the basis of the computed joint reaction force, and determining a joint disorder risk indicator that is an indicator representing a joint disorder risk that is the risk of causing joint disorder, on the basis of the computed feature amount.

(Supplementary note 21) A presymptomatic disease countermeasure method comprising: obtaining motion data that is time-series data representing motion, by measuring the motion of an object, computing joint reaction force at a joint to be evaluated among joints of the object, using the obtained motion data and ground reaction force data that is time-series data representing ground reaction force applied to the object, computing a feature amount representing a load repeatedly applied to the joint to be evaluated on the basis of the computed joint reaction force, determining a joint disorder risk indicator that is an indicator representing a joint disorder risk that is the risk of causing joint disorder, on the basis of the computed feature amount, and outputting the determined joint disorder risk indicator together with countermeasures for preventing development of symptoms caused by the computed joint reaction force.

(Supplementary note 22) A joint disorder risk evaluation program causing a computer to perform: a joint reaction force computation process of computing joint reaction force at a joint to be evaluated among joints of an object, using motion data that is time-series data representing motion of the object, and ground reaction force data that is time-series data representing ground reaction force applied to the object, a feature amount computation process of computing a feature amount representing a load repeatedly applied to the joint to be evaluated on the basis of the computed joint reaction force, and a determination process of determining a joint disorder risk indicator that is an indicator representing a joint disorder risk that is the risk of causing joint disorder, on the basis of the computed feature amount.

(Supplementary note 23) A joint disorder risk evaluation device comprising: a joint reaction force computation unit which computes joint reaction force at a joint whose a joint disorder risk, that is the risk of causing joint disorder, is evaluated among joints of an object, using motion data that is time-series data representing motion of the object, and ground reaction force data that is time-series data representing ground reaction force applied to the object, a feature amount computation unit which computes a feature amount representing a load repeatedly applied to the joint to be evaluated on the basis of the computed joint reaction force, and a determination unit which determines a joint disorder risk indicator that is an indicator representing the joint disorder risk, on the basis of the computed feature amount.

(Supplementary note 24) A joint disorder risk evaluation system comprising: a motion measurement unit which obtains motion data that is time-series data representing motion, by measuring the motion of an object, a joint reaction force computation unit which computes joint reaction force at a joint whose a joint disorder risk, that is the risk of causing joint disorder, is evaluated among joints of the object, using the obtained motion data and ground reaction force data that is time-series data representing ground reaction force applied to the object, a feature amount computation unit which computes a feature amount representing a load repeatedly applied to the joint to be evaluated on the basis of the computed joint reaction force, and a determination unit which determines a joint disorder risk indicator that is an indicator representing the joint disorder risk, on the basis of the computed feature amount.

(Supplementary note 25) A presymptomatic disease countermeasure system comprising: a motion measurement unit which obtains motion data that is time-series data representing motion, by measuring the motion of an object, a joint reaction force computation unit which computes joint reaction force at a joint whose a joint disorder risk, that is the risk of causing joint disorder, is evaluated among joints of the object, using the obtained motion data and ground reaction force data that is time-series data representing ground reaction force applied to the object, a feature amount computation unit which computes a feature amount representing a load repeatedly applied to the joint to be evaluated on the basis of the computed joint reaction force, a determination unit which determines a joint disorder risk indicator that is an indicator representing the joint disorder risk, on the basis of the computed feature amount, and an output unit which outputs the determined joint disorder risk indicator together with countermeasures for preventing development of symptoms caused by the computed joint reaction force.

(Supplementary note 26) A joint disorder risk evaluation method comprising: computing joint reaction force at a joint whose a joint disorder risk, that is the risk of causing joint disorder, is evaluated among joints of an object, using motion data that is time-series data representing motion of the object, and ground reaction force data that is time-series data representing ground reaction force applied to the object, computing a feature amount representing a load repeatedly applied to the joint to be evaluated on the basis of the computed joint reaction force, and determining a joint disorder risk indicator that is an indicator representing the joint disorder risk, on the basis of the computed feature amount.

(Supplementary note 27) A joint disorder risk evaluation method comprising: obtaining motion data that is time-series data representing motion, by measuring the motion of an object, computing joint reaction force at a joint whose a joint disorder risk, that is the risk of causing joint disorder, is evaluated among joints of the object, using the obtained motion data and ground reaction force data that is time-series data representing ground reaction force applied to the object, computing a feature amount representing a load repeatedly applied to the joint to be evaluated on the basis of the computed joint reaction force, and determining a joint disorder risk indicator that is an indicator representing the joint disorder risk, on the basis of the computed feature amount.

(Supplementary note 28) A presymptomatic disease countermeasure method comprising: obtaining motion data that is time-series data representing motion, by measuring the motion of an object, computing joint reaction force at a joint whose a joint disorder risk, that is the risk of causing joint disorder, is evaluated among joints of the object, using the obtained motion data and ground reaction force data that is time-series data representing ground reaction force applied to the object, computing a feature amount representing a load repeatedly applied to the joint to be evaluated on the basis of the computed joint reaction force, determining a joint disorder risk indicator that is an indicator representing the joint disorder risk, on the basis of the computed feature amount, and outputting the determined joint disorder risk indicator together with countermeasures for preventing development of symptoms caused by the computed joint reaction force.

(Supplementary note 29) A joint disorder risk evaluation program causing a computer to perform: a joint reaction force computation process of computing joint reaction force at a joint whose a joint disorder risk, that is the risk of causing joint disorder, is evaluated among joints of an object, using motion data that is time-series data representing motion of the object, and ground reaction force data that is time-series data representing ground reaction force applied to the object, a feature amount computation process of computing a feature amount representing a load repeatedly applied to the joint to be evaluated on the basis of the computed joint reaction force, and a determination process of determining a joint disorder risk indicator that is an indicator representing the joint disorder risk, on the basis of the computed feature amount.

### INDUSTRIAL APPLICABILITY

The present invention is suitably applied to health care systems (especially, a presymptomatic disease countermeasure system for locomotive syndrome) that promote improvement in gait by presenting a joint disorder risk.

In addition, the present invention is also suitably applied to a system that supports the planning of an efficient rehabilitation plan by quantitatively indicating an effect of rehabilitation and a system that objectively computes long-term care insurance premiums or the like by more accurately determining the degree of care.

Further, the present invention is also suitably applied to a system for teaching the running form of healthy persons and athletes, the pitching form of a baseball player, the form of a tennis player or a golf player, etc.

Furthermore, the object of evaluation of the present invention is not limited to humans. For example, the present invention is suitably applied to a system for evaluating a possibility of failure of a joint part of a robot having a joint such as a manipulator, represented by an automobile assembly robot.

### REFERENCE SIGNS LIST

10, 80 joint disorder risk evaluation system
20 pedestrian terminal
30 presymptomatic locomotive syndrome countermeasure system
40 server
50 inputter terminal
60 pedestrian
61 inputter
70, 300 joint disorder risk evaluation device
71, 82, 92 joint reaction force computation unit
72, 83, 93 feature amount computation unit
73, 84, 94 determination unit
81, 91 motion measurement unit
90 presymptomatic disease countermeasure system
95, 306 output unit
100, 100a-100f motion measurement device
200, 200a-200b ground reaction force measurement device
301 CPU
302 main memory unit
303 communication unit
304 auxiliary memory unit
305 input unit
307 system bus
310 dynamics analysis unit
320 feature amount computation unit
330 indicator determination unit
400, 600 storage device
500, 510, 700 display device
610 reference data storage unit
620 presymptomatic disease countermeasure method storage unit
800 input device

What is claimed is:

1. A joint disorder risk evaluation device comprising:
a joint reaction force computation unit which computes joint reaction force at a joint to be evaluated among joints of an object, using motion data that is time-series data representing motion of the object, and ground reaction force data that is time-series data representing ground reaction force applied to the object,
a feature amount computation unit which computes a feature amount representing a load repeatedly applied to the joint to be evaluated on the basis of the computed joint reaction force, and
a determination unit which determines a joint disorder risk indicator that is an indicator representing a joint disorder risk that is the risk of causing joint disorder, on the basis of the computed feature amount.

2. The joint disorder risk evaluation device according to claim 1, wherein
the joint reaction force computation unit uses the motion data obtained from motion measurement means which measures the motion of the object.

3. The joint disorder risk evaluation device according to claim 1, wherein
the joint reaction force computation unit uses the ground reaction force data obtained from ground reaction force measurement means which measures the ground reaction force applied to the object.

4. The joint disorder risk evaluation device according to claim 2, wherein
the joint reaction force computation unit estimates the ground reaction force data on the basis of the obtained motion data, and uses the estimated ground reaction force data.

5. The joint disorder risk evaluation device according to claim 1, wherein
the determination unit determines the joint disorder risk indicator using information indicating correspondence between the feature amount and the joint disorder risk indicator.

6. The joint disorder risk evaluation device according to claim 1, wherein
the joint reaction force computation unit computes a joint moment at the joint to be evaluated and,
the feature amount computation unit computes the feature amount on the basis of the computed joint moment.

7. A joint disorder risk evaluation system comprising:
a motion measurement unit which obtains motion data that is time-series data representing motion, by measuring the motion of an object,
a joint reaction force computation unit which computes joint reaction force at a joint to be evaluated among joints of the object, using the obtained motion data and ground reaction force data that is time-series data representing ground reaction force applied to the object,
a feature amount computation unit which computes a feature amount representing a load repeatedly applied to the joint to be evaluated on the basis of the computed joint reaction force, and
a determination unit which determines a joint disorder risk indicator that is an indicator representing a joint disorder risk that is the risk of causing joint disorder, on the basis of the computed feature amount.

8. The joint disorder risk evaluation system according to claim 7, further comprising a ground reaction force measurement unit which obtains ground reaction force data representing the ground reaction force by measuring the ground reaction force applied to the object,
wherein
the joint reaction force computation unit uses the obtained ground reaction force data.

9. The joint disorder risk evaluation system according to claim 7, wherein
the joint reaction force computation unit estimates the ground reaction force data on the basis of the obtained motion data, and uses the estimated ground reaction force data.

10. The joint disorder risk evaluation system according to claim 7, further comprising a storage unit which stores information indicating correspondence between the feature amount and the joint disorder risk indicator,
wherein the determination unit uses the stored information to determine the joint disorder risk indicator.

11. The joint disorder risk evaluation system according to claim 7, wherein
the joint reaction force computation unit computes a joint moment at the joint to be evaluated and,
the feature amount computation unit computes the feature amount on the basis of the computed joint moment.

12. A presymptomatic disease countermeasure system comprising:
a motion measurement unit which obtains motion data that is time-series data representing motion, by measuring the motion of an object,
a joint reaction force computation unit which computes joint reaction force at a joint to be evaluated among joints of the object, using the obtained motion data and ground reaction force data that is time-series data representing ground reaction force applied to the object,
a feature amount computation unit which computes a feature amount representing a load repeatedly applied to the joint to be evaluated on the basis of the computed joint reaction force,
a determination unit which determines a joint disorder risk indicator that is an indicator representing a joint disorder risk that is the risk of causing joint disorder, on the basis of the computed feature amount, and
an output unit which outputs the determined joint disorder risk indicator together with countermeasures for preventing development of symptoms caused by the computed joint reaction force.

13. The presymptomatic disease countermeasure system according to claim 12, further comprising:
a first storage unit which stores the motion data, the ground reaction force data, and a joint disorder risk indicator as reference data, and
a display unit which displays the stored reference data.

14. The presymptomatic disease countermeasure system according to claim 12, further comprising an input unit to which the countermeasures for preventing development of symptoms are inputted.

15. The presymptomatic disease countermeasure system according to claim 12, further comprising a ground reaction force measurement unit which obtains the ground reaction force data representing the ground reaction force by measuring the ground reaction force applied to the object,
wherein
the joint reaction force computation unit uses the obtained ground reaction force data.

16. The presymptomatic disease countermeasure system according to claim 12, wherein
the joint reaction force computation unit estimates the ground reaction force data on the basis of the obtained motion data, and uses the estimated ground reaction force data.

17. The presymptomatic disease countermeasure system according to claim 12, further comprising a second storage unit which stores information indicating correspondence between the feature amount and the joint disorder risk indicator,
wherein the determination unit uses the stored information to determine the joint disorder risk indicator.

18. The presymptomatic disease countermeasure system according to claim 12, wherein
the joint reaction force computation unit computes a joint moment at the joint to be evaluated and,
the feature amount computation unit computes the feature amount on the basis of the computed joint moment.

19.-22. (canceled)

23. The joint disorder risk evaluation device according to claim 2, wherein
the joint reaction force computation unit uses the ground reaction force data obtained from ground reaction force measurement means which measures the ground reaction force applied to the object.

24. The joint disorder risk evaluation device according to claim 2, wherein
the determination unit determines the joint disorder risk indicator using information indicating correspondence between the feature amount and the joint disorder risk indicator.
